(19) United States
(12) Patent Application Publication
(10) Pub. No.: US 2025/0339386 A1
(43) Pub. Date: Nov. 6, 2025

(54) VITAMIN K2 FOR USE IN TREATMENT OF CORONARY ARTERY CALCIFICATION

(71) Applicants: Region Syddanmark, Vejle (DK); Syddansk Universitet, Odense M (DK)

(72) Inventors: Axel Cosmus Pyndt DIEDERICHSEN, Odense M (DK); Trygve BERGELAND, Oslo (NO)

(21) Appl. No.: 18/869,927

(22) PCT Filed: May 25, 2023
(86) PCT No.: PCT/EP2023/064084

§ 371 Date: Nov. 27, 2024

(30) Foreign Application Priority Data
May 31, 2022 (EP) 22176280.0

(51) Int. Cl. A61K 31/122; A61K 31/592; A61K 31/593; A61P 9/10

(52) U.S. Cl. CPC A61K 31/122; A61K 31/592; A61K 31/593; A61P 9/10

(57) ABSTRACT

The present invention relates to high-dose vitamin K2, in particular menaquinone-7 (MK-7), for use in the treatment of coronary heart disease in high-risk subjects having a coronary artery calcification (CAC) score ≥400 (e.g. at the onset of the treatment), wherein the treatment results in slowing of the progression calcification in the coronary 5 arteries.

### FIELD OF THE INVENTION

The present invention relates to the novel and surprising observation that supplementation with vitamin K2 reduces the progression of further coronary artery calcification (CAC) in patients with severe coronary artery calcification.

### BACKGROUND OF THE INVENTION

Coronary artery calcification (CAC) and progression in CAC is a strong predictor of acute myocardial infarction (AMI) and cardiovascular mortality. Menaquinone-7 (MK-7), which is a type of vitamin K2, is a cofactor for the carboxylation of proteins involved in the inhibition of arterial calcification and has been suggested to reduce the progression rate of aortic valve calcification (AVC) in patients with aortic stenosis.

Moreover, ischaemic heart disease causes 19% and 20% of all deaths among men and women, respectively, thus prevention is of outmost importance. Ischaemic heart disease is often silent until symptoms of myocardial infarction. However, subclinical coronary artery disease is easily detected by non-contrast cardiac CT scans as coronary artery calcifications (CAC). CAC increases with age, and men have higher CAC scores than women. In a population in which CAC is absent, there is a very low risk of future cardiovascular disease (CVD), but as the CAC score increases, so does the risk of ischaemic heart disease. Thus, to prevent CVD, identification and treatment of individuals with severe CAC is important.

Vascular calcification is a slowly progressive process and caused by an imbalance between the mechanisms that promote and inhibit the deposition of calcium in the vessel wall, and vitamin K-dependent proteins play an essential role in this inhibition. The most familiar K vitamin is phylloquinone (vitamin K1), as it is essential in activation of several coagulation factors. Menaquinone (vitamin K2) is another very important vitamin K species. Vitamin K2 is deemed necessary for y-carboxylation of proteins related to the inhibition of arterial calcification, i.e. matrix-Gla proteins (MGP). Without these activated proteins, the balance of cellular calcium uptake and the mineralization process in bone and blood vessels is impaired.

Vitamin K2 enters the circulation through the lymph associated with chylomicrons directed towards the liver. Extrahepatic distribution is facilitated through LDL particles where longer chain menaquinones are shown to have a higher presence compared to shorted chain menaquinones. All tissues that express the LDL receptor will be targeted by vitamin K2, in particular the longer chain menaquinones, like MK-7 (Schurgers, L. J. and C. Vermeer (2002) and Shearer, M. J. and P. Newman (2008)).

In the body there are 20 described vitamin K dependent proteins (VKDP), where vitamin K2 serves as a co-factor to activate these proteins through a carboxylation process (Simes, D. C., et al. (2020)). Matrix-Gla-protein (MGP) is expressed in smooth vascular muscle cells and is a strong inhibitor of vascular calcification. The key to how matrix Gla-protein functions lies with its carboxyl group. Matrix Gla must be carboxylated to function properly, and vitamin K2 functions as a cofactor in this enzymatic reaction together with γ-glutamyl carboxylase. In the presence of vitamin K2, matrix Gla-protein becomes carboxylated, which means it's being turned “on” to repel calcium infiltration. Insufficient vitamin K2 results in matrix Gla being inadequately carboxylated or turned “off,” which means it's unable to inhibit calcium infiltration into soft tissues. One commonly used biomarker for vitamin K2 status in the body is the dephosphorylated uncarboxylated MGP (dp-ucMGP). This biomarker is the inactive form of MGP, and a high level reflects a low vitamin K2 status and vice versa.

Several of the VKDPs display anti-inflammatory functions, like protein C, protein S, Gas 6, and GRP (Simes, D. C., et al. (2020)). In addition there is also data showing anti-inflammatory modulations by vitamin K2 independent of y-glutamyl carboxylase. By using human monocyte-derived macrophages, Pan and colleagues showed inhibition of cytokine release (TNF-α, IL-1α, IL-1β) when the cells were pretreated by vitamin K2 (menaquinone-7), and the scientists were able to show a dose response relationship (Pan, M. H., et al. (2016)). Similar inhibitory effects are also seen on IL-6, by other research groups (Ohsaki, Y., et al. (2006) and Reddi, K., et al. (1995)). The release of pro-inflammatory cytokines is mainly regulated through the NF-kB signaling pathway, and vitamin K is shown to inhibit the release of IkB from NF-kB to allow its entry into the nucleus (Ohsaki, Y., et al. (2010), Ozaki, I., et al. (2009), Xia, J., et al. (2012)).

Statins are widely used due to their lipid-lowering effects and the prevention of cardiovascular events. In 2015, a controversial hypothesis was presented where the action of statins not only inhibited the synthesis of cholesterol, but through the inhibition of HMG-CoA reductase also inhibited prenyl-intermediate levels and thereby also inhibiting the conversion of vitamin K1 to K2 in the body (Okuyama, H., et al. (2015)). Since vitamin K2 has a higher preference for the LDL particles directed towards the extrahepatic tissues, the use of statins could lead to an extrahepatic vitamin K deficiency resulting in less activation of the VKDPs in different tissues. A cross-sectional clinical trial investigated the connection between statin use, CAC and activation levels of VKDPs. The authors found a higher CAC score among the statin users, they did not find any difference in the carboxylation status of MGP, however, they found a significantly higher level of the uncarboxylated form of the VKDP Osteocalcin among statin users (Zhelyazkova-Savova, M. D., et al. (2021)).

Currently, there are no recommendations of vitamin K2 supplementation available. Also, it is well-known that the daily intake of vitamin K2 in the Western world is not sufficient to meet the request for a complete activation of MGP. Additionally, there is no documented toxicity for vitamin K1 or vitamin K2, and the WHO has set no upper tolerance level for vitamin K intake.

The effect of high-dose vitamin K2 supplementation on aortic valve calcification progression was examined in the recent AVADEC trial, published in Diederichsen A C P et al. (2022). Said publication discloses the treatment of male subjects with aortic valve calcification score above 300 arbitrary units. In said study, however, aortic valve calcification progression was non-significantly decreased.

EP1728507 A1 discloses in studies of rats that high intake of vitamin K can lead to removal of calcified precipitates from blood vessels that have already been affected by pre-existing calcification.

EP2558084 B1 discloses in studies of rats a pharmaceutical composition comprising a vitamin K component and a nicotinamide component for use in the prevention or treatment of a disorder which accompanies extraosseous calcification.

WO19021232 A1 relates to a composition comprising a K-group vitamin, or analogues and derivatives thereof, an inorganic magnesium salt and an iron (III) oxide, complex or salt, —pharmaceutical or food grade excipients, additives and/or co-formulants for use in a preventive or curative method for treating vascular calcification in in vitro experiments (experimental model consisting of calcified vascular smooth muscle cells removed from rat aorta).

WO19191773 A1 discloses administration into mammals of vitamin K, including vitamin K2, to quickly reverse calcification of blood vessels. WO19191773 A1 further discloses a clinical study carried out on kidney transplantation patients in order to study the efficacy and safety of Vitamin K2 supplementation on arterial stiffness.

EP1728507 B1 discloses inter alia the use of vitamin K2, e.g. combined with vitamin D, for reversing calcification of a blood vessel in a rat model.

Geleijnse J. M. et al. (2004) discloses inter alia the effects of dietary menaquinone in relation to aortic calcification and coronary heart disease (CHD) in men and women aged 55 years and over without prior history of myocardial infarction at baseline.

Shea M. K. et al. (2009) discloses inter alia the effects of vitamin K1 (phylloquinone) supplementation on CAC progression in older men and women between 60-80 years of age, without known coronary heart disease (asymptomatic) at baseline and concludes that vitamin K1 supplementation reduced the progression of existing CAC in asymptomatic older men and women when taken with recommended amounts of calcium and vitamin D.

None of the above prior art documents disclose or suggest vitamin K2 administration or supplementation in subjects (e.g. human patients) with severe CAC, defined by a CAC-score of ≥400, i.e. subjects being in high-risk of developing acute myocardial infarction (AMI).

Hence, providing a treatment for reducing the progression of further coronary artery calcification in a patient sub-group with severe CAC, defined by a CAC-score of ≥400 would be advantageous.

### SUMMARY OF THE INVENTION

The prevalence of CAC score above 400 in the Danish population was previously studied in in a population-based study (Diederichsen A. C. P. et al. (2012)), showing that 2% of 50 years old men had CAC score above 400, while increasing to 14% of 60 years old men. 1% of 50 years old women had CAC score above 400, while increasing to 4% of 60 years old women. Furthermore, among randomly selected Danish men and women aged 65-74 years a CAC score >400 was found in 37.8% of men and 11.3% of women (Kvist T. V. et al. (2017)).

The present invention was conceived in this context and is based on the surprising observation that supplementation with vitamin K2 reduces the progression of CAC in subjects (e.g. human subjects) with severe CAC as defined by a CAC-score ≥400, when compared to placebo. In particular, high-dose supplementation with vitamin K2 and vitamin D reduces the progression of CAC in subjects (e.g. human subjects) with severe CAC as defined by a CAC-score ≥400.

Thus, an object of the present invention relates to treating this particular subgroup of subjects (e.g. patients), defined by having a CAC-score ≥400, determined by cardiac CT scanning and by using the so-called Agatston method or a similar method for determination of CAC.

In a first aspect, the invention provides vitamin K2 for use in the prevention or treatment of coronary heart disease in a subject having a coronary artery calcification (CAC) score of ≥400.

The invention also relates to vitamin K2 for use in the prevention or treatment of coronary heart disease in a subject having a CAC-score of ≥400, by administration to said subject of 100 μg-100 mg vitamin K2 per day, preferably 200 μg-50 mg vitamin K2 per day, preferably 360 μg-10 mg vitamin K2 per day, preferably 360 μg-5 mg vitamin K2 per day, preferably 360-1440 μg vitamin K2 per day, preferably 500-1000 μg vitamin K2 per day, more preferably 720 μg vitamin K2 per day, preferably wherein the vitamin K2 is combined with administration of vitamin D, preferably 1-50 μg vitamin D per day, 10-50 μg vitamin D per day, preferably 25 μg vitamin D per day, e.g. wherein the prevention or treatment results in slowing of the progression of calcification in the coronary arteries.

In a another aspect, the invention provides vitamin K2 for use in slowing of the progression of calcification in the coronary arteries.

In still a another aspect, the invention provides vitamin K2 for use in slowing of the progression of calcification in the coronary arteries, wherein the slowing of the progression of calcification in the coronary arteries is a reduction in CAC score determined by cardiac CT scanning and by using the Agatston method or a similar method for determination of CAC by at least 1%, at least 2%, at least 3%, at least 4%, at least 5%, at least 6%, at least 7%, at least 8.9%.

In still a another aspect, the invention provides vitamin K2 for use in slowing of the progression of calcification in the coronary arteries, wherein the slowing of the progression of calcification in the coronary arteries is a reduction of progression of calcified plaque (mm³) in intervention group by at least 1%, at least 3%, at least 9%, at least 12%, and at least 12.7%.

In still a another aspect, the invention provides vitamin K2 for use in slowing of the progression of calcification in the coronary arteries, wherein the slowing of the progression of calcification in the coronary arteries is a reduction of progression of noncalcified plaque (mm³) in intervention group by at least 1%, at least 3%, at least 5%, at least 7%, and at least 8%.

In still a another aspect, the invention provides vitamin K2 for use in reduction of calcification in the coronary arteries as measured by CAC score determined by cardiac CT scanning and by using the Agatston method or a similar method for determination of CAC by at least 0.5%, at least 1%, at least 1.5%, at least 2%, at least 2.5%, at least 4.8% when compared to baseline.

In still a another aspect, the invention provides vitamin K2 for use in reduction of total plaque volume in the coronary arteries of at least, at least 1%, at least 2%, at least 3%, at least 4%, at least 5%, at least 6%, at least 7%, at least 8%, at least 9%, at least 10%, at least 11%, at least 12%, at least 13.1% when compared to baseline.

In another aspect, the invention provides a method of treating coronary heart disease in a subject having a CAC-score ≥400, wherein the method involves administration of vitamin K2 to said subject

Another aspect of the present invention relates to a method of preventing or treating calcification in the coronary arteries in a subject having a CAC-score of ≥400, wherein the method involves administration of vitamin K2 to said subject.

The present invention may relate to a method of treating calcification in the coronary arteries of a subject having a CAC-score of ≥400, by administration to said subject between 100 μg-100 mg vitamin K2 per day, preferably 200 μg-50 mg vitamin K2 per day, preferably 360 μg-10 mg vitamin K2 per day, preferably 360 μg-5 mg vitamin K2 per day, preferably 360-1440 μg vitamin K2 per day, preferably 500-1000 μg vitamin K2 per day, preferably 720 μg vitamin K2 per day, preferably combined with administration of vitamin D, preferably with 1-50 μg vitamin D per day, preferably 10-50 μg vitamin D per day, preferably 25 μg vitamin D per day.

Another aspect relates to the use of vitamin K2 in the manufacture of a medicament for the prevention or treatment of coronary heart disease in a subject having a coronary artery calcification (CAC) score ≥400.

Another aspect relates to a kit of parts comprising
(A) vitamin K2, and
(B) vitamin D;
   for use in the prevention or treatment of coronary heart disease in a subject having a coronary artery calcification (CAC) score ≥400.

### BRIEF DESCRIPTION OF THE FIGURES

FIG. 1 shows the progression of CAC in both the intervention and placebo group from baseline to 24 month follow-up (203 AU vs. 254 AU, p=0.089) for all tested subjects (total cohort).

FIG. 2 shows the progression of CAC a subgroup with a baseline CAC score <400 AU. Among subjects with a baseline CAC score <400 AU there were no difference in progression (77 AU versus 81 AU, p=0.849).

FIG. 3 shows the progression of CAC in a subgroup with a baseline CAC score ≥ 400 AU. Among subjects with a baseline CAC score ≥ 400 AU there was a decreased progression in CAC score at 24 months follow-up (288 AU versus 380 AU, p=0.047).

FIG. 4 shows plaque composition in the cohort. Specifically, FIG. 4 shows the total plaque composition divided in two subgroups: “Calcified plaque burden” and “Non-calcified plaque burden”. The latter is further characterized in “Low-attenuation plaque burden”. (see table 3 for further details)

FIG. 5 shows that following a 24 months supplementation treatment with vitamin K2 (K2VITAL® Delta tablet, 720 μg/day) supplemented by the recommended daily dose of vitamin D (25 μg/day) in form of orally administered tablets, the placebo group had a mean 36.95 pmol/L increase of dp-ucMGP versus −229,4 pmol/L in the intervention group (P<0.0001).

FIGS. 6a-c show that following a 24 months supplementation treatment with vitamin K2 (K2VITAL® Delta tablet, 720 μg/day) supplemented by the recommended daily dose of vitamin D (25 μg/day) in form of orally administered tablets, at baseline, there was no difference between patients on statins vs patients not on statins with regards to carboxylation status of dp-ucMGP (FIG. 6a). In the placebo group, there was no difference between patients on statins vs patients not on statins with regards to carboxylation status of dp-ucMGP during the supplementation period (FIG. 6b). In the intervention group, the non-statin users had a mean −203.2 pmol/L reduction of dp-ucMGP vs −239.8 pmol/L in the statin group (FIG. 6c).

FIG. 7 shows that following a 24 months supplementation treatment with vitamin K2 (K2VITAL® Delta tablet, 720 μg/day) supplemented by the recommended daily dose of vitamin D (25 μg/day) in form of orally administered tablets, progression of non-calcified plaque volume was 46 mm³ in the placebo group versus −6 mm³ in the intervention group (p=0.172). Progression of calcified plaque volume was 20 mm³ in the placebo group versus 2 mm³ in the intervention group (p=0.179). Progression of total plaque volume was 66 mm³ in the placebo group versus −4 mm³ in the intervention group (p=0.146).

FIGS. 8a-b show that following a 24 months supplementation treatment with vitamin K2 (K2VITAL® Delta tablet, 720 μg/day) supplemented by the recommended daily dose of vitamin D (25 μg/day) in form of orally administered tablets, for the whole population, from Baseline to 24 months, in the placebo group 7 out of 132 had a reduction in CAC score vs 13 out of 143 in the intervention group (p=0.2533) (FIG. 8a). Among patients with a CAC score ≥400, from Baseline to 24 months, in the placebo group 3 out of 76 had a reduction in CAC score vs 10 out of 85 in the intervention group (p=0.0858) (FIG. 8b).

FIG. 9 shows that following a 24 months supplementation treatment with vitamin K2 (K2VITAL® Delta tablet, 720 μg/day) supplemented by the recommended daily dose of vitamin D (25 μg/day) in form of orally administered tablets, a number of patients had a reduction in total plaque volume from Baseline to 24 months. In the placebo group 23 out of 98 had a reduction in total plaque volume vs 34 out of 109 in the intervention group

The present invention will now be described in more detail in the following.

### DETAILED DESCRIPTION OF THE INVENTION

Prior to discussing the present invention in further details, the following terms and conventions will first be defined:

Agatston method: as used herein, the system for the quantification of the CAC score in CT images, is the Agatston method, which uses the weighted sum of lesions with a density above 130 HU (Hounsfield scale), multiplying the area of calcium by a factor related to maximum plaque attenuation: 130-199 HU, factor 1; 200-299 HU, factor 2; 300-399 HU, factor 3; and ≥400 HU, factor 4 (see e.g. Neves P O et al. (2017) which is incorporated herein by reference). The Agatston method is well known in the art.

CAC score: as used herein CAC refers to “coronary artery calcification”. To determine a CAC score, a non-contrast CT-scan has to be performed. The CAC score reflects the amount of calcium in the walls of the heart's arteries. This test uses a special type of imaging test called a computed tomography (CT) scan of the heart. This scan produces multiple pictures to check if calcium is present and, if so, how much. A calcium score is calculated based on the amount of plaque observed in the CT scan (quantified by the Agatston method) (see also e.g. Neves P O et al. (2017)). The CAC score is well known in the art.

Vitamin K2: as used herein, vitamin K2 refers to any of the vitamin K2 homologues, menaquinone-4 (MK-4), menaquinone-5 (MK-5), menaquinone-6 (MK-6), menaquinone-7 (MK-7), menaquinone-8 (MK-8), menaquinone-9 (MK-9), menaquinone-10 (MK-10), menaquinone-11 (MK-11), menaquinone-12 (MK-12) or menaquinone-13 (MK-13), or combinations thereof. It is the number n of isoprenyl units in their side chain differs and ranges from 4 to 13, hence Vitamin K2 consists of various forms. It is indicated as a suffix (-n), e. g. MK-7 contains seven isoprenyl units.

By vitamin K2 is also meant prodrugs of vitamin K2. Suitable prodrugs are described in, for example, WO2013/128037.

Coronary heart disease may herein be referred to as ischaemic heart disease or coronary artery disease.

### Embodiments of the Invention

One embodiment of the invention relates to vitamin K2 for use in the prevention or treatment of, or a method of preventing or treating, coronary heart disease in a subject having a coronary artery calcification (CAC) score ≥400 as determined by cardiac CT scanning and by using the Agatston method or a similar method for determination of CAC. Typically, the prevention or treatment described herein results in slowing of the progression of calcification in the coronary arteries.

In the embodiments and aspects of the invention, where a CAC score of ≥400 is referred to, it is typically meant a CAC score of ≥400 at the onset of the treatment. As is discussed in the Examples, the beneficial effects are seen in subjects who show high CAC levels to start with.

The vitamin K2 (e.g. MK-7) may be administered to the subject at a dosage of 100 μg-100 mg vitamin K2 per day, preferably 200 μg-50 mg vitamin K2 per day, preferably 360 μg-10 mg vitamin K2 per day, preferably 360 μg-5 mg vitamin K2 per day, preferably 360-1440 μg vitamin K2 per day, preferably 500-1000 μg vitamin K2 per day, most preferably 720 μg vitamin K2 per day. Other suitable dosages for vitamin K2 include at least 100 μg vitamin K2 per day, preferably at least 360 μg vitamin K2 per day, e.g. at least 500 μg vitamin K2 per day.

Another embodiment of the invention therefore relates to vitamin K2 for use in the treatment of, or a method of treating, coronary heart disease in a subject having a coronary artery calcification (CAC) score ≥400 (e.g. at the onset of the treatment) as determined by cardiac CT scanning and by using the Agatston method or a similar method for determination of CAC, wherein the subject is administered with 100 μg-100 mg vitamin K2 per day, preferably 200 μg-50 mg vitamin K2 per day, preferably 360 μg-10 mg vitamin K2 per day, preferably 360 μg-5 mg vitamin K2 per day, preferably 360-1440 μg vitamin K2per day, preferably 500-1000 μg vitamin K2 per day, most preferably 720 μg vitamin K2 per day. Other suitable dosages for vitamin K2 include at least 100 μg vitamin K2 per day, preferably at least 360 μg vitamin K2 per day, e.g. at least 500 μg vitamin K2 per day.

Still another embodiment of the invention relates to vitamin K2 for use in the treatment of, or a method of treating, coronary heart disease in a subject having a coronary artery calcification (CAC) score ≥400 (e.g. at the onset of the treatment) as determined by cardiac CT scanning and by using the Agatston method or a similar method for determination of CAC, wherein the subject is administered with between 100 μg-100 mg vitamin K2 per day, preferably 200 μg-50 mg vitamin K2 per day, preferably 360 μg-10 mg vitamin K2 per day, preferably 360 μg-5 mg vitamin K2 per day, preferably 360-1440 μg vitamin K2 per day, preferably 500-1000 μg vitamin K2 per day, most preferably 720 μg vitamin K2 per day, administered in combination with vitamin D, preferably with between 1-50 μg vitamin D per day, preferably 10-50 μg vitamin D per day, preferably 25 μg vitamin D per day, preferably wherein the treatment results in slowing of the progression calcification in the coronary arteries.

Still another embodiment of the invention relates to vitamin K2 for use in the prevention or treatment of coronary heart disease in a subject having a coronary artery calcification (CAC) score of ≥400 determined by cardiac CT scanning and by using the Agatston method or a similar method for determination of CAC, wherein the subject is having a blood concentration of dephosphorylated uncarboxylated matrix-Gla-protein (dp-ucMGP) of 433 to 2179 pmol/L, 433 to 500 pmol/L, 475 to 600 pmol/L, 550 to 700 pmol/L, 650 to 800 pmol/L, 750 to 900 pmol/L, 850 to 1000 pmol/L, 950 to 1250 pmol/L, 1200 to 1750 pmol/L, 1700 to 2000 pmol/L, and 1950 to 2179 pmol/L.

Still another embodiment of the invention relates to vitamin K2 for use in the prevention or treatment of coronary heart disease in a subject having a coronary artery calcification (CAC) score of ≥400 determined by cardiac CT scanning and by using the Agatston method or a similar method for determination of CAC, wherein the subject is having a blood concentration of dephosphorylated uncarboxylated matrix-Gla-protein (dp-ucMGP) of 433 to 2179 pmol/L, 400 to 2000 pmol/L preferably 425 to 1500 pmol/L, preferably 450 to 1250 pmol/L, preferably 475 to 1000 pmol/L, more preferably 500 to 900 pmol/L.

In a another aspect, the invention provides vitamin K2 for use in slowing of the progression of calcification in the coronary arteries.

In still a another aspect, the invention provides vitamin K2 for use in slowing of the progression of calcification in the coronary arteries, wherein the slowing of the progression of calcification in the coronary arteries is a reduction in CAC score determined by cardiac CT scanning and by using the Agatston method or a similar method for determination of CAC by at least 1%, at least 2%, at least 3%, at least 4%, at least 5%, at least 6%, at least 7%, at least 8.9%.

In still a another aspect, the invention provides vitamin K2 for use in slowing of the progression of calcification in the coronary arteries, wherein the slowing of the progression of calcification in the coronary arteries is a reduction of progression of calcified plaque (mm³) in intervention group by at least 1%, at least 3%, at least 9%, at least 12%, and at least 12.7%.

In still a another aspect, the invention provides vitamin K2 for use in slowing of the progression of calcification in the coronary arteries, wherein the slowing of the progression of calcification in the coronary arteries is a reduction of progression of noncalcified plaque (mm³) in intervention group by at least 1%, at least 3%, at least 5%, at least 7%, and at least 8%.

In still a another aspect, the invention provides vitamin K2 for use in reduction of calcification in the coronary arteries as measured by CAC score determined by cardiac CT scanning and by using the Agatston method or a similar method for determination of CAC by at least 0.5%, at least 1%, at least 1.5%, at least 2%, at least 2.5%, at least 4.8% when compared to baseline.

In still a another aspect, the invention provides vitamin K2 for use in reduction of total plaque volume in the coronary arteries of at least, at least 1%, at least 2%, at least 3%, at least 4%, at least 5%, at least 6%, at least 7%, at least 8%, at least 9%, at least 10%, at least 11%, at least 12%, at least 13.1% when compared to baseline.

Still another embodiment of the invention relates to a method of preventing or treating coronary heart disease in a subject having a CAC-score ≥400, as determined by cardiac CT scanning and by using the Agatston method or a similar method for determination of CAC, wherein the method involves administration of vitamin K2 to said subject.

Still another embodiment of the invention relates to a method of preventing or treating coronary heart disease in a subject having a CAC-score ≥400, as determined by cardiac CT scanning and by using the Agatston method or a similar method for determination of CAC, wherein the method involves administration of vitamin K2 to said subject, wherein the vitamin K2 is administered at a dose of 100 μg-100 mg vitamin K2 per day, preferably 200 μg-50 mg vitamin K2 per day, preferably 360 μg-10 mg vitamin K2 per day, preferably 360 μg-5 mg vitamin K2 per day, preferably 360-1440 μg vitamin K2 per day, preferably 500-1000 μg per day, more preferably 720 μg vitamin K2 per day.

Still another embodiment of the invention relates to a method of preventing or treating coronary heart disease in a subject having a CAC-score ≥400, as determined by cardiac CT scanning and by using the Agatston method or a similar method for determination of CAC, wherein the method involves administration of vitamin K2 to said subject, wherein the vitamin K2 is administered at a dose of 720 μg vitamin K2 per day. wherein vitamin K2 is administered in combination with vitamin D, preferably with between 1-50 μg vitamin D per day, preferably 10-50 μg vitamin D per day, preferably 25 μg vitamin D per day.

Still another embodiment of the invention relates to a method of preventing or treating coronary heart disease in a subject having a CAC-score >400, as determined by cardiac CT scanning and by using the Agatston method or a similar method for determination of CAC, wherein the method involves administration of vitamin K2 to said subject, wherein the vitamin K2 is administered at a dose of 720 μg vitamin K2 per day. wherein vitamin K2 is administered in combination 25 μg vitamin D per day, wherein vitamin K2 is selected from any of the vitamin K2 homologues, menaquinone-4 (MK-4), menaquinone-5 (MK-5), menaquinone-6 (MK-6), menaquinone-7 (MK-7), menaquinone-8 (MK-8), menaquinone-9(MK-9), menaquinone-10 (MK-10), menaquinone-11 (MK-11), menaquinone-12 (MK-12) or menaquinone-13 (MK-13), or combinations thereof.

Still another embodiment of the invention relates to a method of preventing or treating coronary heart disease in a subject having a CAC-score ≥400, as determined by cardiac CT scanning and by using the Agatston method or a similar method for determination of CAC, wherein the method involves administration of vitamin K2 to said subject, wherein the vitamin K2 is administered at a dose of 720 μg vitamin K2 per day. wherein vitamin K2 is administered in combination 25 μg vitamin D per day. wherein vitamin K2 is menaquinone-7 (MK-7).

Still another embodiment of the invention relates to a method of preventing or treating coronary heart disease in a subject having a CAC-score ≥400, as determined by cardiac CT scanning and by using the Agatston method or a similar method for determination of CAC, wherein the method involves administration of vitamin K2 to said subject, wherein the vitamin K2 is administered at a dose of 720 μg vitamin K2 per day. wherein vitamin K2 is administered in combination 25 μg vitamin D per day. wherein vitamin K2 is menaquinone-7 (MK-7), wherein vitamin K2 is administered as an injectable or oral formulation.

Still another embodiment of the invention relates to a method of slowing of the progression of calcification in the coronary arteries in a subject by administering vitamin K2, preferably 720 μg vitamin K2 per day in the form of menaquinone-7 (MK-7), into said subject.

Still another embodiment of the invention relates to a method of reducing the total non-calcified plaque volume in the coronary arteries in a subject by administering vitamin K2, preferably 720 μg vitamin K2 per day in the form of menaquinone-7 (MK-7), into said subject.

Still another embodiment of the invention relates to a method of reducing the blood concentration of dephosphorylated uncarboxylated matrix-Gla-protein (dp-ucMGP) in a subject by administering vitamin K2, preferably 720 μg vitamin K2 per day in the form of menaquinone-7 (MK-7), into said subject.

Still another embodiment of the invention relates to a method of reducing cardiovascular events, such as myocardial infarction, revascularization and death in a subject with no previous myocardial infarction, percutaneous coronary intervention or coronary artery bypass graft surgery, by administering vitamin K2, preferably 720 μg vitamin K2 per day in the form of menaquinone-7 (MK-7), into said subject.

The vitamin K2 to be administered according to the present invention may be any of the vitamin K2 homologues, menaquinone-4 (MK-4), menaquinone-5 (MK-5), menaquinone-6 (MK-6), menaquinone-7 (MK-7), menaquinone-8 (MK-8), menaquinone-9 (MK-9), menaquinone-10 (MK-10), menaquinone-11 (MK-11), menaquinone-12 (MK-12) or menaquinone-13 (MK-13), or combinations thereof, preferably menaquinone-7 (MK-7).

The vitamin D to be administered according to an embodiment of the present invention may be any of vitamin D1, vitamin D2, vitamin D3, vitamin D4 or vitamin D5, or combinations thereof. The vitamin D is typically administered in dosages of 1-50 μg per day, preferably 10-50 μg vitamin D per day, more preferably 25 μg vitamin D per day. Other suitable ranges for vitamin D administration include at least 1 μg vitamin D per day, preferably at least 5 μg vitamin D per day, preferably at least 10 μg vitamin D per day.

The vitamin K2 (and optionally the vitamin D) may be administered as a pharmaceutically acceptable composition, e.g. containing at least one excipient. The vitamin K2 may also be administered in the form of a microencapsulated product, as described in WO2015/169816 for example.

According to another embodiment of the invention, both the vitamin K2 source and/or the vitamin D source can be administered to the subject (e.g. the patient) as an injectable or oral formulation. Oral administration/formulation of vitamin K2 and vitamin D is preferred.

If both vitamin K2 and vitamin D are administered, these can be administered simultaneously, separately or sequentially. This applies to all embodiments/aspects of the invention, whether compounds for use, kits for use, methods, uses, etc.

The compounds or combinations of the invention can be used on any animal subject, in particular a mammal and more particularly on a human or an animal serving as a model for a disease (e.g. mouse, monkey, etc.), preferably a human. Preferably, the subject is a human subject, therefore.

The invention also provides a combination product comprising vitamin K2 and vitamin D, for use in the prevention or treatment of coronary heart disease in a subject having a coronary artery calcification (CAC) score ≥400 (e.g. at the onset of the treatment).

The invention also provides a kit of parts (e.g. a pharmaceutical kit of parts) comprising
(A) vitamin K2, and
(B) vitamin D;
for use in the prevention or treatment of coronary heart disease in a subject having a coronary artery calcification (CAC) score ≥400 (e.g. at the onset of the treatment), e.g. wherein the treatment results in slowing of the progression of calcification in the coronary arteries. The two parts of the kit ((A) and (B)) may be in the form of separate oral formulations, or separate injectable formulations, for example.

According to still another embodiment of the invention, the total period of administering the vitamin K2 and/or vitamin D in the dosages according to the present invention is between 0.1-15 years, preferably 0.5-10 years, more preferably 1-5 years, most preferably 2 years. Other suitable periods include at least 0.1 years, e.g. at least 0.5 years, e.g. at least 1 year, e.g. at least 2 years.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1 (Non-Published Sub-Study of the So-Called AVADEC Trial)

The present invention is based on a sub-study of the so-called AVADEC trial. In said sub-study, the inventors examined the progression of CAC in participants with no prior coronary disease (no myocardial infarction and/or revascularization) at baseline.

The change in CAC was evaluated in the entire group and in two prespecified subgroups (low-risk: CAC score <400 AU and high-risk: CAC ≥400 AU at baseline).

304 participants (male, mean age 71 years) with no prior coronary disease and with aortic valve calcification score ≥300 were randomized (1:1) to treatment with vitamin K2 (K2VITAL® Delta tablet, 720 μg/day) supplemented by the recommended daily dose of vitamin D (25 μg/day) in form of orally administered tablets or placebo treatment (no active treatment) for two years. K2VITAL®Delta tablet is an MK-7 tablet formulation. Exclusion criteria are treatment with vitamin K antagonist or coagulation disorders.

Non-contrast CT-scans were performed at baseline (0 months), 12 and 24 months of follow-up. Contrast CT-scans were performed at baseline (0 months) and 24 months. CAC score was measured with established software and expressed in AU. On contrast CT-scans, quantitative coronary plaque composition evaluations were performed by using Autoplaque. For further details, see Example 2.

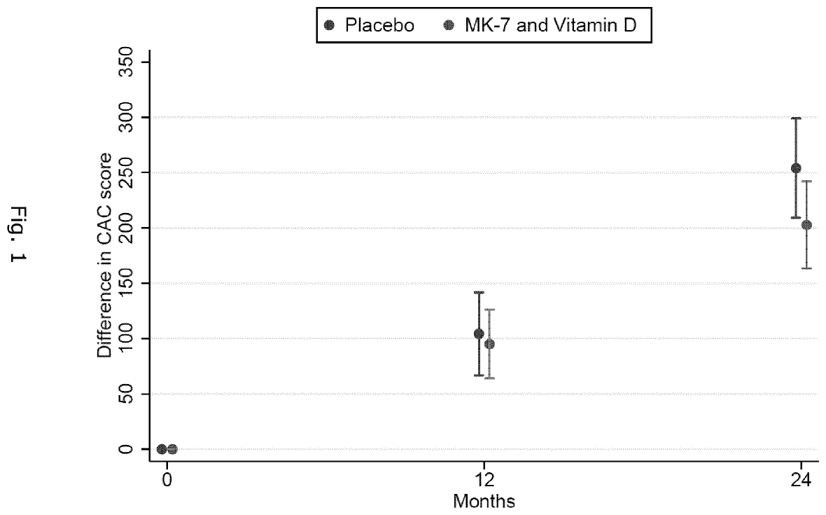

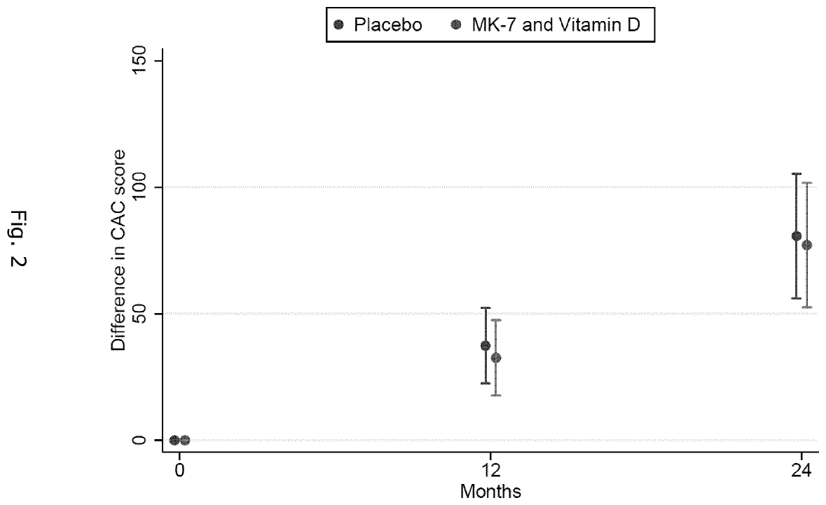

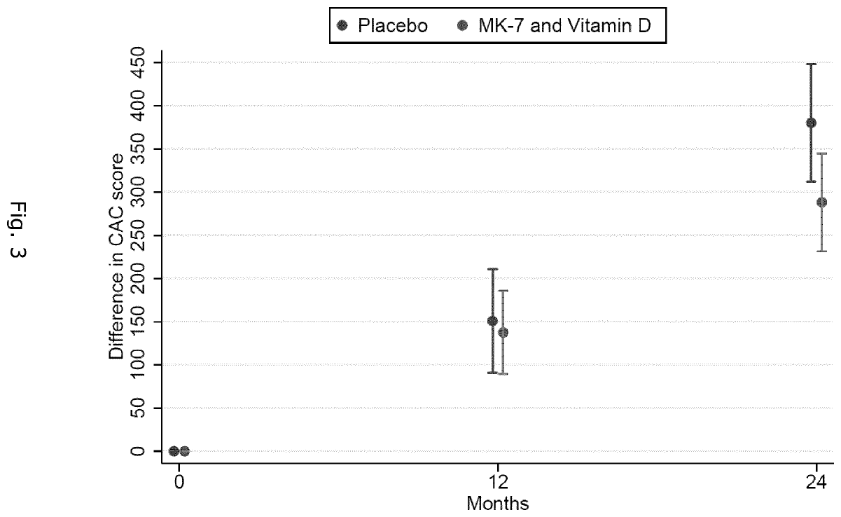

The intervention and placebo groups were similar in all traditional cardiovascular risk factors except familial predisposition for cardiovascular disease (14.4% vs. 6.7%, p=0.046). The inventors found progression of CAC in both the intervention and placebo group from baseline (0 months) to 24 month follow-up (203 AU vs. 254 AU, p=0.089) (cf. FIGS. 1-3).

The patients with CAC score <400 AU at baseline (0 months) were equal in progression (77 AU vs. 81 AU, p=0.846). In patients with CAC score ≥400, the progression of CAC was significantly lower in the intervention group (288 AU vs. 380 AU, p=0.047). Yet, preliminary analyses of contrast CT-scans in 180 participants showed no difference in the progression of non-calcified plaque volume (10 mm³ vs. 37 mm³, p=0.276). In addition, the number of events (all-cause mortality, myocardial infarction and coronary revascularization) was significantly lower in participants receiving vitamin K2 and D (1.9% vs. 6.7%, p=0.048).

TABLE 1a
Characteristics of patients at baseline (0 months)

| | | | Vitamin K2 | | |
|---|---|---|---|---|---|
| Factor | Placebo tablets | | and D tablets | | p-value |
| N | 149 | | 155 | | |
| Age, mean (SD) | 71.16 (2.24) | | 70.75 (6.12) | | 0.44 |
| | (n = 149) | | (n = 155) | | |
| BMI, median (IQR) | 28.00 (26.00, | | 29.00 (26.00, | | 0.75 |
| | 31.00) (n = 142) | | 32.00) (n = 145) | | |
| High blood pressure | 95 | (63.8%) | 105 | (67.7%) | 0.46 |
| Diabetes | 22 | (14.8%) | 28 | (18.1%) | 0.44 |
| Atrial fibrillation | 17 | (11.5%) | 15 | (9.7%) | 0.61 |
| Renal failure | 11 | (7.4%) | 21 | (13.6%) | 0.080 |
| Hereditary disposition | 10 | (6.7%) | 22 | (14.4%) | 0.046 |
| Tobacco | | | | | |
| Never smoker | 46 | (30.9%) | 48 | (31.4%) | 0.83 |
| Former smoker | 83 | (55.7%) | 88 | (57.5%) | |
| Current smoker | 20 | (13.4%) | 17 | (11.1%) | |
| P-HDL, mean (SD) | 1.36 (0.33) (n = 140) | | 1.37 (0.39) (n = 145) | | 0.74 |
| P-LDL, mean (SD) | 2.27 (0.86) (n = 140) | | 2.31 (0.89) (n = 145) | | 0.66 |
| P-cholesterol, | 4.25 (1.01) (n = 140) | | 4.32 (1.00) (n = 145) | | 0.51 |
| mean (SD) | | | | | |
| eGFR, median (IQR) | 81.00 (70.00, 88.00) | | 81.00 (67.00, 89.00) | | 0.48 |
| | (n = 148) | | (n = 154) | | |
| Systolic blood | 148.00 (135.00, | | 144.00 (133.00, | | 0.13 |
| pressure, median | 161.00) (n = 146) | | 154.00) (n = 153) | | |
| (IQR) | | | | | |
| Diastolic blood | 86.50 (79.00, | | 83.00 (78.00, | | 0.032 |
| pressure, median | 94.00) (n = 146) | | 90.00) (n = 153) | | |
| (IQR) | | | | | |
| Treatment with | 79 | (53.0%) | 80 | (51.6%) | 0.81 |
| ACE/AT2 | | | | | |
| Treatment with | 26 | (17.4%) | 32 | (20.6%) | 0.48 |
| Beta-blockers | | | | | |
| Treatment with | 8 | (5.4%) | 4 | (2.6%) | 0.21 |
| Ca-saving diuretics | | | | | |
| Treatment with | 96 | (64.4%) | 100 | (64.5%) | 0.99 |
| trombocyt inhibitors | | | | | |
| AK-treatment | 14 | (9.4%) | 14 | (9.0%) | 0.91 |
| Treatment with | 109 | (73.2%) | 106 | (68.4%) | 0.36 |
| statins | | | | | |
| Agatston score, | 654.70 (181.60, | | 636.40 (200.10, | | 0.95 |
| median (IQR) | 1380.00) (n = 149) | | 1443.30) (n = 155) | | |
| CAC <400 | 60 | (40.3%) | 62 | (40.0%) | 0.96 |
| CAC ≥400 | 89 | (59.7%) | 93 | (60.0%) | |

TABLE 1b
Characteristics of CAC score at baseline (0 months)

| | | Min | Max | Mean | Median |
|---|---|---|---|---|---|
| BASELINE | N | (AU) | (AU) | (AU) | (AU) |
| Whole population | 304 | 0 | 6269 | 1034 | 641.5 |
| Placebo group | 149 | 0 | 5687 | 1031 | 654.7 |
| Intervention group | 155 | 0 | 6269 | 1037 | 636.4 |

Table 2. Progression of CAC Score. Mean Change in CAC Score from 0 to 24 Months

TABLE 2a
Entire group

| | | (intervention) | (difference) | |
|---|---|---|---|---|
| | Placebo | Vitamin K2 + D | Vitamin − placebo | |
| Outcome | 0-24 m | 0-24 m | 0-24 m | p-value |
| Δ CAC | 253.95 (209.07; | 202.68 (163.31; | −51.27 (−110.36; | 0.089 |
| score | 298.83) | 242.05) | 7.83) | |

CAC score increased by 253.95 in the placebo group, while it increased by 202.68 in the vitamin K2 + D group

TABLE 2b
Subgroup with baseline CAC score below 400

| | | (intervention) | (difference) | |
|---|---|---|---|---|
| | Placebo | Vitamin K2 + D | Vitamin − placebo | |
| Outcome | 0-24 m | 0-24 m | 0-24 m | p-value |
| Δ CAC | 80.71 (56.08; | 77.12 (52.49; | −3.59 (−39.80; | 0.846 |
| score | 105.35) | 101.76) | 32.62) | |

CAC score increased by 80.71 in the placebo group, while it increased by 77.12 in the vitamin K2 + D group. There was no significant difference.

TABLE 2c
Subgroup with baseline CAC score ≥400

| | | (intervention) | (difference) | |
|---|---|---|---|---|
| | Placebo | Vitamin K2 + D | Vitamin − placebo | |
| Outcome | 0-24 m | 0-24 m | 0-24 m | p-value |
| Δ CAC | 379.95 (311.86; | 288.07 (231.41; | −91.88 (−182.52; | 0.047 |
| score | 448.03) | 344.73) | −1.23) | |

CAC score increased by 379.95 in the placebo group, while it increased by 288.07 in the vitamin K2 + D group. This corresponds to a significantly lower progression in CAC score at 91.88 in the intervention group.

TABLE 3
Analyses on plaque composition in the total cohort

| | | (intervention) | (difference) | |
|---|---|---|---|---|
| | Placebo | Vitamin K2 − D | vitamin − placebo | |
| Outcome | 0-24 m | 0-24 m | 0-24 m | p-value |
| Total calcified plaque | 14.56 (0.33; | 13.77 (3.42; | −0.79 (−18.47; | 0.930 |
| volume, mm³ | 28.80) | 24.13) | 16.89) | |
| Total low density | −0.55 (−14.42; | 4.61 (−9.27; | 5.15 (−14.12; | 0.600 |
| non-calcified plaque | 13.33) | 18.49) | 24.43) | |
| volume, mm³ | | | | |
| Total non- | 37.10 (1.92; | 10.37 (−21.81; | −26.74 (−74.87; | 0.276 |
| calcified plaque | 72.28) | 42.55) | 21.40) | |
| volume, mm³ | | | | |
| Total plaque | 51.96 (6.09; | 23.89 (−11.84; | −28.08 (−87.22; | 0.352 |
| volume, mm³ | 97.84) | 59.62) | 31.07) | |

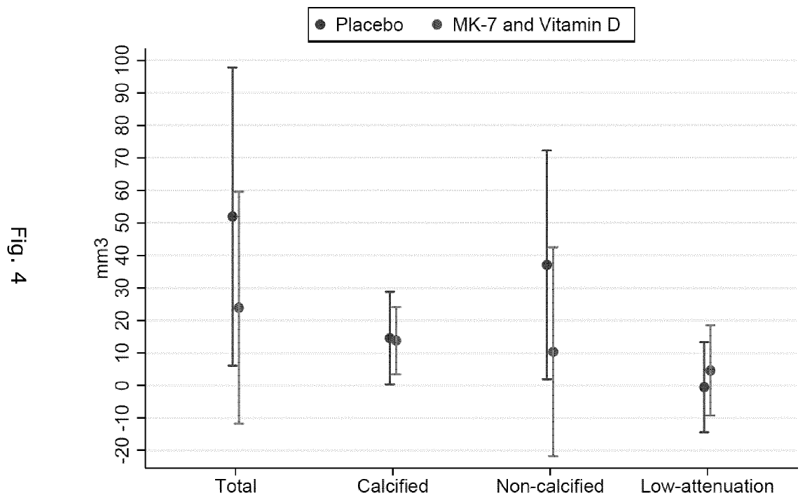

The analyses in Table 3 are not divided in CAC score <400 versus CAC score ≥400. For further details, see FIG. 4.

### Example 2 (Determination of CAC-Score by Multi-Slice Computed Tomography Scans)

Cardiac CT scans was performed using a dedicated cardiac CT-scanner. A standard non-contrast as well as contrast scan is performed according to usual clinical care.

To assess the CAC scores the following CT settings are used: 120 kV tube voltage, and a prospectively scan 300 ms after the QRS-complex. The scanning protocol during the contrast scan depends on the local CT scanner and the patient heart rate. In patients with a stable heart rate above 60 beats per minute (bpm), orally or intravenously β-blocker are administered until the heart rate is appropriate (if possible below 60), and a prospectively gated protocol is used. In patients with a heart rate >70 bpm despite β-blocker pretreatment or in case of an irregular heart rhythm, a prospectively scan 200-400 ms after the QRS-complex is performed.

Additionally, sublingual nitrates are administered prior to the scan. 50-80 mL of contrast agent are injected into an antecubital vein at a rate of 6.0 mL/s followed by 60 mL intravenous saline (6.0 mL/s) using a dual-head power injector. Data acquisition parameters depends on the local CT scanner, but slice collimation will be below 0.6 mm, gantry rotation time as fast as possible and a tube voltage of 70 or 120 kV depending on patients' weight.

All scans are sent to and analyzed at the core lab, Odense University Hospital. CAC scores are measured by using the Agatston method, i.e. by summing-up all spots of calcifications in the coronaries.

The coronary artery tree will be analyzed for the presence and severity of coronary artery disease (CAD), according to the classification of the American Heart Association 16-segment model. Coronary plaques are defined as visible structures within or adjacent to the coronary artery lumen, which can be clearly distinguished from the vessel lumen and the surrounding pericardial tissue. All coronary segments ≥2 mm in diameter with plaque will be analyzed using a semi-automated software. Scans were analyzed by an experienced cardiologist.

### Example 3 (Vitamin K2 Status Measured by dp-ucMGP)

For the whole population, the carboxylation status of dp-ucMGP ranged from 387 pmol/L (min) to 2179 pmol/L (max) with a mean of 773 pmol/L and a median of 732 pmol/L. In the placebo group, dp-ucMGP ranged from 387 pmol/L (min) to 1323 pmol/L (max) with a mean of 763 pmol/L and a median of 718 pmol/L. In the intervention group, dp-ucMGP ranged from 456 pmol/L (min) to 2179 pmol/L (max) with a mean of 783 pmol/L and a median of 736 pmol/L.

For patients with a CAC score ≥400, the carboxylation status of dp-ucMGP ranged from 433 pmol/L (min) to 2179 pmol/L (max) with a mean of 789 pmol/L and a median of 746 pmol/L. In the placebo group, dp-ucMGP ranged from 433 pmol/L (min) to 1323 pmol/L (max) with a mean of 769 pmol/L and a median of 728 pmol/L. In the intervention group, dp-ucMGP ranged from 456 pmol/L (min) to 2179 pmol/L (max) with a mean of 808 pmol/L and a median of 751 pmol/L.

TABLE 4

| | | Min | Max | Mean | Median |
|---|---|---|---|---|---|
| BASELINE | N | (pmol/L) | (pmol/L) | (pmol/L) | (pmol/L) |
| Whole population | 285 | 387 | 2179 | 773 | 732 |
| Placebo group | 140 | 387 | 1323 | 763 | 718 |
| Intervention group | 145 | 456 | 2179 | 783 | 736 |
| CAC ≥400, | 172 | 433 | 2179 | 789 | 746 |
| both groups | | | | | |
| CAC ≥400, | 83 | 433 | 1323 | 769 | 728 |
| Placebo group | | | | | |
| CAC ≥400, | 89 | 456 | 2179 | 809 | 751 |
| Intervention group | | | | | |

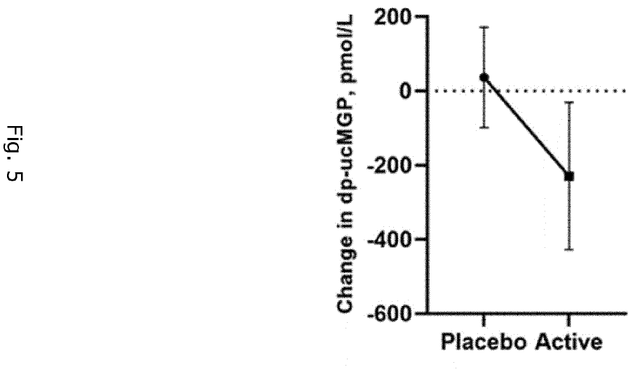

Following a 24 months supplementation, the placebo group had a mean 36.95 pmol/L increase of dp-ucMGP versus −229,4 pmol/L in the intervention group (P<0.0001), (see FIG. 5).

TABLE 5

| | Placebo | Intervention/ | Group difference | |
|---|---|---|---|---|
| | group | Active group | Difference (95% CI) | p-value |
| Change of | 36.95 (137) | −229.4 (121) | −266.3 (−309; −224) | P < 0.0001 |
| dp-ucMGP, | | | | |
| pmol/L (N) | | | | |

### Example 4 (Use of Statins)

In a stratified analysis looking into treatment-by-subgroup interaction, participants on statin treatment had a significant reduction of CAC score progression by intervention (p=0.048).

TABLE 6

| | | Intervention/ | | |
|---|---|---|---|---|
| | Placebo group; | Active group | | |
| | Mean change in | Mean change in | Treatment effect | |
| | CAC score from | CAC score from | Difference | |
| | Baseline (95% CI) | Baseline (95% CI) | (95% CI) | p-value |
| Statin | 301 (243; 358) | 225 (178; 272) | −76 (−151; −1) | P = 0.048 |
| therapy | | | | |

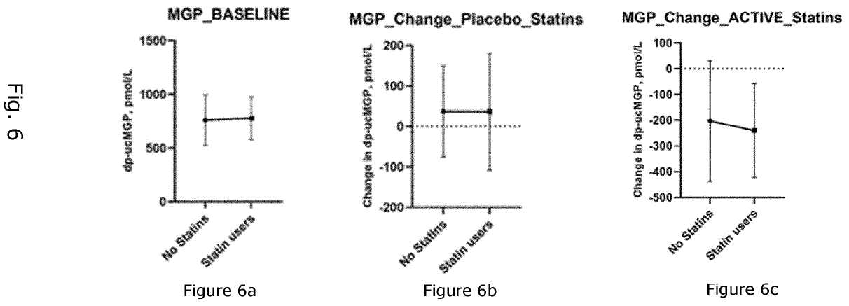

At Baseline, there was no difference between patients on statins vs patients not on statins with regards to carboxylation status of dp-ucMGP (FIG. 6a). In the placebo group, there was no difference between patients on statins vs patients not on statins with regards to carboxylation status of dp-ucMGP during the supplementation period (FIG. 6b). In the intervention group, the non-statin users had a mean −203.2 pmol/L reduction of dp-ucMGP vs −239.8 pmol/L in the statin group (FIG. 6c).

TABLE 7

| | No statin | | Statin | | Group difference | | |
|---|---|---|---|---|---|---|---|
| | users | | users | | Difference (95% CI) | | p-value |
| Dp-ucMGP at | 760.9 | (81) | 777.7 | (204) | 16.75 | (−38; 71) | p = 0.5449 |
| Baseline, | | | | | | | |
| pmol/L (N) | | | | | | | |
| Placebo group, | 37.4 | (34) | 36.8 | (87) | −0.56 | (−55; 54) | p = 0.9838 |
| change of dp-ucMGP, | | | | | | | |
| pmol/L (N) | | | | | | | |
| Intervention group, | −203.2 | (39) | −239.8 | (98) | −36.58 | (−111; 38) | p = 0.3318 |
| change of dp-ucMGP, | | | | | | | |
| pmol/L (N) | | | | | | | |

### Example 5 (Plaque Volume and Composition)

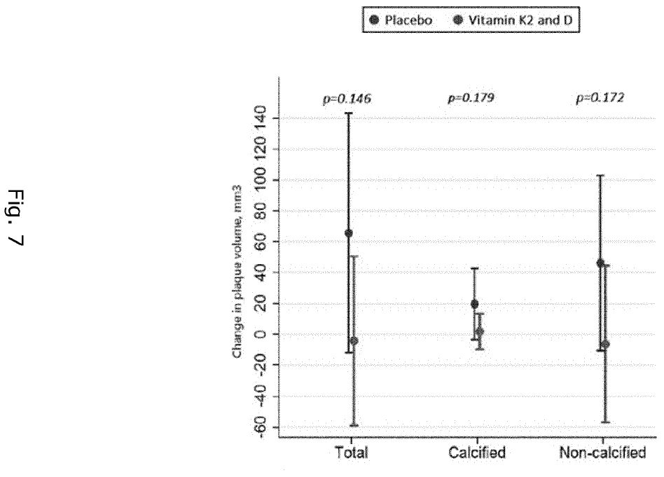

Plaque volume and composition was measured for the whole cohort by contrast CT. Only images with good and excellent (at both Baseline and 24 months) were included in the analysis. Progression of non-calcified plaque volume was 46 mm³ in the placebo group versus −6 mm³ in the intervention group (p=0.172). Progression of calcified plaque volume was 20 mm³ in the placebo group versus 2 mm³ in the intervention group (p=0.179). Progression of total plaque volume was 66 mm³ in the placebo group versus −4 mm³ in the intervention group (p=0.146) (see FIG. 7).

TABLE 8
(Total Plaque volume at Baseline)

| BASELINE - images | | | | | |
|---|---|---|---|---|---|
| with good and | | Min | Max | Mean | Median |
| excellent quality | N | (mm³) | (mm³) | (mm³) | (mm³) |
| Whole group | 101 | 25 | 2949 | 804 | 660 |
| Placebo group | 45 | 25 | 2949 | 826 | 689 |
| Intervention group | 56 | 29 | 2562 | 786 | 576 |

TABLE 9
(Calcified plaque volume at Baseline)

| BASELINE - images | | | | | |
|---|---|---|---|---|---|
| with good and | | Min | Max | Mean | Median |
| excellent quality | N | (mm³) | (mm³) | (mm³) | (mm³) |
| Whole group | 101 | 0 | 900 | 142 | 76 |
| Placebo group | 45 | 3 | 900 | 157 | 80 |
| Intervention group | 56 | 0 | 866 | 130 | 69 |

TABLE 10
(Non-calcified plaque volume at Baseline)

| BASELINE - images | | | | | |
|---|---|---|---|---|---|
| with good and | | Min | Max | Mean | Median |
| excellent quality | N | (mm³) | (mm³) | (mm³) | (mm³) |
| Whole group | 101 | 20 | 2114 | 662 | 558 |
| Placebo group | 45 | 20 | 2114 | 669 | 600 |
| Intervention group | 56 | 27 | 1942 | 656 | 484, 5 |

TABLE 11

| Placebo group | Vitamin K + D | | |
|---|---|---|---|
| (N = 40) | group (N = 46) | Group difference | |
| Mean change | Mean change | Treatment | |
| from baseline | from baseline | effect | |
| (95% CI) | (95% CI) | (95% CI) | p-value |

Plaque volume and composition

| | | | | | | | |
|---|---|---|---|---|---|---|---|
| Total plaque | 66 | (−12; 143) | −4 | (−59; 51) | −70 | (−164; 24) | 0.146 |
| (mm³) | | | | | | | |
| Calcified | 20 | (−4; 43) | 2 | (−10; 13) | −18 | (−44; 8) | 0.179 |
| plaque (mm³) | | | | | | | |
| Noncalcified | 46 | (−11; 103) | −6 | (−57; 45) | −53 | (−128; 23) | 0.172 |
| plaque (mm³) | | | | | | | |

### Example 6 (Reductions in CAC Score and Total Plaque Volume)

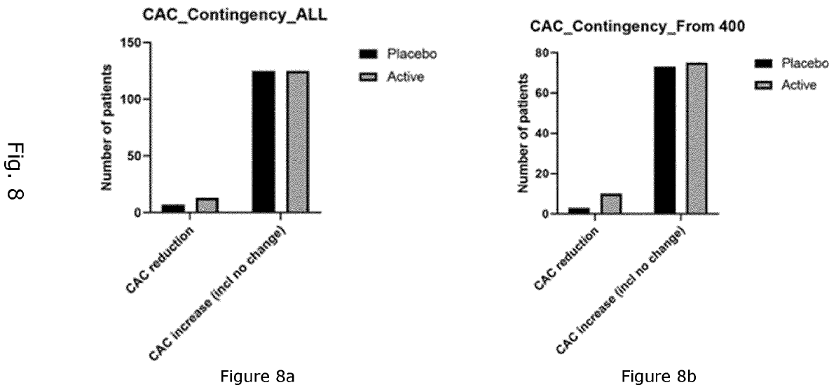

A categorical analysis was performed where the number of patients that had a reduction in CAC score from Baseline to 24 months were counted. For the whole population, in the placebo group 7 out of 132 had a reduction in CAC score vs 13 out of 143 in the intervention group (p=0.2533). Among patients with a CAC score ≥400, in the placebo group 3 out of 76 had a reduction in CAC score vs 10 out of 85 in the intervention group (p=0.0858) (see FIG. 8a-b).

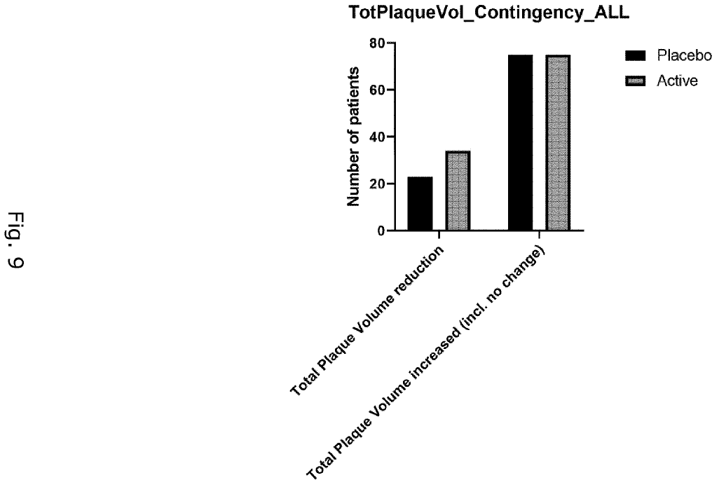

Furthermore, a categorical analysis was performed where the number of patients that had a reduction in total plaque volume from Baseline to 24 months were counted (all image qualities). In the placebo group 23 out of 98 had a reduction in total plaque volume vs 34 out of 109 in the intervention group (p=0.2753) (see FIG. 9).

### Example 7 (Cardiovascular Event Rate)

A total of 13 participants had a clinical safety event during the follow-up period. The event rate (combined event rate of myocardial infarctions, revascularizations, and all-cause death) was 10 (6.7%) in the placebo group versus 3 (1.9%) in the vitamin K2 and D group (p=0.048).

TABLE 12

| | | |
|---|---|---|
| Placebo | Vitamin K + | Group difference |

| | group | D group | Numerical | |
|---|---|---|---|---|
| | Number (%) | Number (%) | difference | p-value |
| Events | 10 (6.7%) | 3 (1.9%) | 7 | 0.048 |

### References

Diederichsen A. C. P. et al. (2012): (DanRisk study) “Discrepancy between coronary artery calcium score and HeartScore in middle-aged Danes: the DanRisk study”, (Eur J Prev Cardiol. 2012 June; 19(3):558-64)

Diederichsen A. C. P. et al. (2022): “Vitamin K2 and D in Patients With Aortic Valve Calcification: A Randomized Double-Blinded Clinical Trial”, Circulation. 25 Apr. 2022; 145:1387-1397.

Geleijnse J. M. et al. (2004): “Dietary intake of menaquinone is associated with reduced risk of coronary heart disease: The Rotterdam study”, American Society for Nutritional Sciences: 3100-3105.

Kvist T. V. et al. (2017): (DANCAVAS pilot study), “The DanCavas Pilot Study of Multifaceted Screening for Subclinical Cardiovascular Disease in Men and Women Aged 65-74 Years”, Eur J Vasc Endovasc Surg (2017) 53, 123-131.

Ohsaki, Y., et al., Vitamin K suppresses the lipopolysaccharide-induced expression of inflammatory cytokines in cultured macrophage-like cells via the inhibition of the activation of nuclear factor kappaB through the repression of IKKalpha/beta phosphorylation. J Nutr Biochem, 2010. 21(11): p. 1120-6.

Okuyama, H., et al., Statins stimulate atherosclerosis and heart failure: pharmacological mechanisms. Expert Rev Clin Pharmacol, 2015. 8(2): p. 189-99.

Ozaki, I., et al., Menatetrenone, a vitamin K2 analogue, inhibits hepatocellular carcinoma cell growth by suppressing cyclin D1 expression through inhibition of nuclear factor kappaB activation. Clin Cancer Res, 2007. 13(7): p. 2236-45.

Pan, M. H., et al., Inhibition of TNF-alpha, IL-1alpha, and IL-1beta by Pretreatment of Human Monocyte-Derived Macrophages with Menaquinone-7 and Cell Activation with TLR Agonists In Vitro. J Med Food, 2016. 19(7): p. 663-9.

Schurgers, L. J. and C. Vermeer, Differential lipoprotein transport pathways of K-vitamins in healthy subjects. Biochim Biophys Acta, 2002. 1570(1): p. 27-32.

Shea M. K. et al. (2009): “Vitamin K supplementation and progression of coronary artery calcium in older men and women”, Am J Clin Nutr 2009; 89:1799-1807.

Shearer, M. J. and P. Newman, Metabolism and cell biology of vitamin K. Thromb Haemost, 2008. 100(4): p. 530-47.

Simes, D. C., et al., Vitamin K as a Diet Supplement with Impact in Human Health: Current Evidence in Age-Related Diseases. Nutrients, 2020. 12(1).

Xia, J., et al., The role of PKC isoforms in the inhibition of NF-kappaB activation by vitamin K2 in human hepatocellular carcinoma cells. J Nutr Biochem, 2012. 23(12): p. 1668-75.

Zhelyazkova-Savova, M. D., et al., Statins, vascular calcification, and vitamin K-dependent proteins: Is there a relation? Kaohsiung J Med Sci, 2021. 37(7): p. 624-631.

1. Vitamin K2 for use in the prevention or treatment of coronary heart disease in a subject having a coronary artery calcification (CAC) score of ≥400 determined by cardiac CT scanning and by using the Agatston method or a similar method for determination of CAC.

2. Vitamin K2 for use in the prevention or treatment of coronary heart disease in a subject having a blood concentration of dephosphorylated uncarboxylated matrix-Gla-protein (dp-ucMGP) of 433 to 2179 pmol/L.

3. Vitamin K2 for use according to claim 2, wherein the blood concentration of dephosphorylated uncarboxylated matrix-Gla-protein (dp-ucMGP) is 433 to 500 pmol/L, 475 to 600 pmol/L, 550 to 700 pmol/L, 650 to 800 pmol/L, 750 to 900 pmol/L, 850 to 1000 pmol/L, 950 to 1250 pmol/L, 1200 to 1750 pmol/L, 1700 to 2000 pmol/L, and 1950 to 2179 pmol/L.

4. Vitamin K2 for use according to claim 2, wherein the blood concentration of dephosphorylated uncarboxylated matrix-Gla-protein (dp-ucMGP) is 400 to 2000 pmol/L preferably 425 to 1500 pmol/L, preferably 450 to 1250 pmol/L, preferably 475 to 1000 pmol/L, more preferably 500 to 900 pmol/L.

5. Vitamin K2 for use in the prevention or treatment of coronary heart disease in a subject on statin treatment.

6. Vitamin K2 for use according to any of claims 1-5, wherein the subject is administered with between 100 μg-100 mg vitamin K2 per day, preferably 200 μg-50 mg vitamin K2 per day, preferably 360 μg-10 mg vitamin K2 per day, preferably 360 μg-5 mg vitamin K2 per day, preferably 360-1440 μg vitamin K2 per day, preferably 500-1000 μg per day, more preferably 720 μg vitamin K2 per day.

7. Vitamin K2 for use according to any of claims 1-6, wherein vitamin K2 is administered in combination with vitamin D, preferably between 1-50 μg vitamin D per day, more preferably between 10-50 μg vitamin D per day, most preferably 25 μg vitamin D per day.

8. Vitamin K2 for use according to any of claims 1-7, wherein vitamin K2 is selected from any of the vitamin K2 homologues, menaquinone-4 (MK-4), menaquinone-5 (MK-5), menaquinone-6 (MK-6), menaquinone-7 (MK-7), menaquinone-8 (MK-8), menaquinone-9 (MK-9), menaquinone-10 (MK-10), menaquinone-11 (MK-11), menaquinone-12 (MK-12) or menaquinone-13 (MK-13), or combinations thereof.

9. Vitamin K2 for use according to any of claims 1-8, wherein the vitamin D to be administered in combination with the vitamin K2 is selected from any of Vitamin D1, Vitamin D2, Vitamin D3, Vitamin D4 or Vitamin D5, or combinations thereof.

10. Vitamin K2 for use according to any of claims 1-9, wherein vitamin K2 is administered as an injectable or oral formulation.

11. Vitamin K2 for use in slowing of the progression of calcification in the coronary arteries.

12. Vitamin K2 for use according to claim 11, wherein the slowing of the progression of calcification in the coronary arteries is a reduction in CAC score determined by cardiac CT scanning and by using the Agatston method or a similar method for determination of CAC by at least 1%, at least 2%, at least 3%, at least 4%, at least 5%, at least 6%, at least 7%, at least 8.9%.

13. Vitamin K2 for use according to claim 11, wherein the slowing of the progression of calcification in the coronary arteries is a reduction of progression of calcified plaque (mm³) in intervention group by at least 1%, at least 3%, at least 9%, at least 12%, and at least 12.7%.

14. Vitamin K2 for use according to claim 11, wherein the slowing of the progression of calcification in the coronary arteries is a reduction of progression of noncalcified plaque (mm³) in intervention group by at least 1%, at least 3%, at least 5%, at least 7%, and at least 8%.

15. Vitamin K2 for use in reduction of calcification in the coronary arteries as measured by CAC score determined by cardiac CT scanning and by using the Agatston method or a similar method for determination of CAC by at least 0.5%, at least 1%, at least 1.5%, at least 2%, at least 2.5%, at least 4.8% when compared to baseline.

16. Vitamin K2 for use in reduction of total plaque volume in the coronary arteries of at least 1%, at least 2%, at least 3%, at least 4%, at least 5%, at least 6%, at least 7%, at least 8%, at least 9%, at least 10%, at least 11%, at least 12%, at least 13.1% when compared to baseline.
